(19) United States
(12) Patent Application Publication
(10) Pub. No.: US 2007/0218166 A1
(43) Pub. Date: Sep. 20, 2007

(54) Test Kit and Method for the Determination of Nitrogen Components in Wine

(76) Inventors: Dieter Tanzer, Rossdorf (DE); Marianne Bauer, Gross-Greau (DE)

Correspondence Address: Millen ,White , Zelan & branigan , P.C., 2200 Clarendon Boulevare, Suite 1400, Arlington, VA, 22201, US

(21) Appl. No.: 11/578,434

(22) PCT Filed: Mar. 26, 2005
(86) PCT No.: PCT/EP05/03236

§ 371 Date: Oct. 17, 2006

(30) Foreign Application Priority Data
Apr. 21, 2004 (EP) 04009399.9

(51) Int. Cl. G01N 31/22; G01N 33/14

(52) U.S. Cl. 426/11; 435/287.1

(57) ABSTRACT

The invention relates to a test kit based on a dry-chemical determination method and to the method for the rapid determination of the content of yeast-available nitrogen components in must and wine. By means of the method according to the invention, nitrogen is determined in the form of ammonium. It is possible here to determine on the one hand free ammonium and on the other hand ammonium which has previously been liberated from arginine.

The invention relates to a test kit based on a dry-chemical determination method and to the method for the rapid determination of the content of yeast-available nitrogen components in must and wine.

The total nitrogen content of grape must is dependent on many factors, in particular on viticultural factors, such as, for example, the location (soil, microclimate), the soil cultivation and fertilisation, the vine variety, the state of health and state of ripening of the grape material and the harvest (time, technique). It is thus highly season—but also enormously weather-dependent. Production-related factors, such as, for example, mash treatment, must treatment and pressing technique, are of secondary importance.

The total nitrogen content is on average 100 to 500 mg/l

According to literature data (Bergner, Lemperle: Weinkompendium [Wine Compendium] (1998) Verlag Hirzel, Stuttgart, and Ullmann, Enzyklopädie der technischen Chemie [Encyclopaedia of Industrial Chemistry] (1983), Volume 24, Chapter on Wine; Verlag Chemie, Weinheim), it is distributed approximately as indicated below:

| Protein N | 30 to 40 | mg/l |
|---|---|---|
| Amino acids N | 30-200 | mg/l |
| Ammonium N | 20-200 | mg/l |

However, the yeasts employed for winemaking do not always find in grape musts the necessary nutrients which are necessary for an optimum fermentation process.

For their multiplication and metabolic processes, the yeast can only utilise ammonium and some amino acids. Not every amino acid is equally valuable for the yeast here. Arginine and proline are the principal amino acids in grape must. While the amino acid arginine is very valuable for the yeast owing to its three accessible nitrogen atoms, the amino acid proline is completely unusable for the yeast. Higher-molecular-weight nitrogen compounds, such as proteins, are not taken up by the yeast. (Würdig, Wohler: Chemie des Weines, Handbuch der Lebensmitteltechnologie [Chemistry of Wine, Handbook of Food Technology] (1989) Verlag Eugen Ulmer, Stuttgart; R. Amann, J. Sigler, H. Krebs; Der Badische Winzer, August 2001, pp. 30-33).

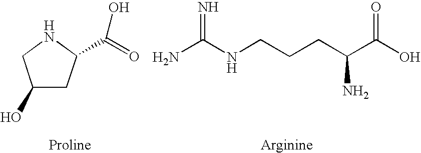

With regard to optimum wine quality, it is vital for readily utilisable nitrogen to be available in sufficient quantity to the yeast for its own nutrition during fermentation. Knowledge of the nitrogen supply is the basis for taking suitable measures, such as, for example, the addition of N fertilisers in the form of ammonium phosphate.

In order to measure the yeast-available nitrogen, the following methods have been employed to date (R. Amann, J. Sigler, H. Krebs; Der Badische Winzer, August 2001, pp. 30-33):

a) Specific Determination of Ammonium and Individual Amino Acids:

These determinations are very complex in equipment terms and require a lot of time, which is naturally associated with high costs.

b) Determination of the Total Nitrogen Content:

The total nitrogen content of a must is significantly higher than the yeast-available nitrogen since the nitrogen from peptides, proteins and the amino acid proline is also included. It is thus not a sensible measurement criterion for evaluation of the nutrient supply.

In the determination, the must is firstly boiled with concentrated sulfuric acid, during which all N compounds are converted into ammonium. The ammonium content is then determined titrimetrically after neutralisation and distillation. This determination requires very considerable effort and the determination is not very reproducible.

c) Determination of the Formol Number:

In the determination of the formol number, all amino groups (NH2) of the amino acids, but not the three additional nitrogen atoms of arginine, are included in addition to the free ammonium. In particular in the case of musts having high arginine contents, a significant part of the yeast-available nitrogen is thus not present, while some of the proline nitrogen, which is not useful for the yeast, is included. The result obtained in the formol number determination is only a numerical value and not a concentration indication of the nitrogen content.

Although the titration on which the determination is based is not very complex in equipment terms, it is, however, necessary to handle formaldehyde, which is harmful to health.

d) NOPA Method:

The name is derived from a reaction of nitrogen (N) with the reagent OPA (ortho-phthalaldehyde). By means of a photometric measurement, all NH₂ groups of the amino acids are included. Free ammonium and the additional nitrogen groups in arginine are not included in the determination, and consequently this method allows only little information regarding the nutrient supply.

e) Infrared Spectroscopic Determination (C.-D- Patz, A. Giehl, H. Dietrich; Der Deutsche Weinbau, 20, 2000, pp. 30-33)

As a multidetection method, infrared spectroscopy has been taken up by large laboratories which carry out a very large number of analyses. With the aid of liquid FTIR, it is possible to analyse musts or wines without sample preparation (if necessary after filtration) in a few minutes for the majority of the significant parameters. In order to achieve reliable quantitative measurement results, however, complex calibration measurements are vital. In addition, quantification in the lower ppm range still causes major difficulties, which is why this method has still not found acceptance.

f) Geisenheimer Test Kit from Erbsloh, Drinks Technology:

In this photometric enzymatic determination, the amino acid arginine, which represents one of the two principal amino acids in grape musts, is determined selectively in addition to free ammonium. This determination method gives a good overview of the nutrient supply of a grape must, is the most widely recognised of all methods and is commercially available as a finished test combination (Erbsloh, Geisenheim). However, it is extremely complicated and time-consuming to carry out and requires a photometer.

A common feature of all methods is that the determination is very complex and time consuming. The determination can only be carried out by expert personnel and requires corresponding instrument equipment. Rapid checking of the N supply by untrained personnel with the aim of immediate decision making regarding process control was hitherto impossible. In addition, the shelf life of the commercially available tests in accordance with Example f) is very short at 6 weeks. All determinations (about 20 per test) must therefore be carried out in a short time (at most within 6 weeks).

Analysis using solid, sorptive supports, so-called test sticks, has increasingly gained in importance recently. The essential advantages of these dry-chemical methods include, in particular, simple handling and straightforward disposal owing to the small amounts of reagents. All or the great majority of the reagents necessary for the determination reaction are embedded in corresponding layers of a solid, sorptive or swellable support, to which the sample is applied. After contact of the reaction zone with the sample, the determination reaction proceeds. The colour formed is a measure of the amount of the analyte to be determined and can be evaluated visually, i.e. semi-quantitatively, or quantitatively using simple reflectometers.

Test sticks for the determination of ammonium in aqueous solutions are commercially available (for example Merck MQ or RQ ammonium test, Merck KGaA, Darmstadt). For the analysis, the test sticks are dipped into the sample.

The main disadvantage of these test sticks is that low ammonium contents, as are present in must, either cannot be determined sufficiently or the complex and acidic (pH about 2.8-3.5) wine matrix interferes with the determination. Use of coloured samples, such as, for example, red wines, is only possible to a limited extent due to the interference by the inherent colour of the sample, since the test sticks come directly into contact with the sample liquid to be investigated.

No tests based on text strips are available for determination of individual amino acids.

The present invention is based on the object of providing a simple method for the determination of yeast-available nitrogen in must/wine samples which does not have the above disadvantages, is simple and quick to carry out, is inexpensive and has a good shelf life in the form of a test kit. In particular, the method according to the invention should be accessible not only to semi-quantitative, visual evaluation, but also to quantitative evaluation using a reflectometer.

It has been found that the determination of the yeast-available nitrogen supply on the basis of the determination of free ammonium and the determination of the sum of free ammonium and the ammonium additionally liberated from the amino acid arginine (referred to below as total ammonium) is indeed possible simply and quickly if use is made of a correspondingly pretreated test stick with which the analysis can be carried out in the gas phase above the sample. It has been observed that the ammonium content in the gas phase above the sample can be employed as a measure of the concentration of ammonium in the sample.

The present invention therefore relates to a test kit for the determination of yeast-available nitrogen in must and wine, at least consisting of a test strip for the determination of ammonia in the gas phase and a sealable vessel. The test kit preferably contains a plurality of test strips and a plurality of analytical vessels.

In a preferred embodiment, the test strip of the kit contains potassium iodide and mercury iodide as determination reagent.

The test kit particularly preferably contains a test strip which has been impregnated with 0.5 to 5% solutions of potassium iodide and mercury iodide.

In another preferred embodiment, the test kit additionally contains one or more of the following components:
0.1 mol/l NaOH solution
32% NaOH solution
alkaline buffer solution for dilution of the sample solution
the two enzymes arginase and urease in dissolved form.

The invention also relates to a method for the determination of yeast-available nitrogen in must and wine, characterised by the following method steps:
a) provision of a test kit according to the invention
b) addition of the sample solution to the analytical vessel,
c) optionally addition of the enzymes arginase and urease in order to liberate ammonium from arginine, and incubation,
d) alkalinisation of the sample solution in order to expel the ammonium as ammonia,
e) introduction of the moistened test stick into the gas space above the sample and sealing of the analytical vessel,
f) qualitative and/or quantitative analysis of the colour development on the test strip.

In a preferred embodiment, a test stick moistened with 0.1 mol/l NaOH solution is employed in step e).

In another preferred embodiment, the sample is alkalinised with 32% NaOH solution in step d).

In a further preferred embodiment, the quantitative analysis in step f) is carried out using a reflectometer.

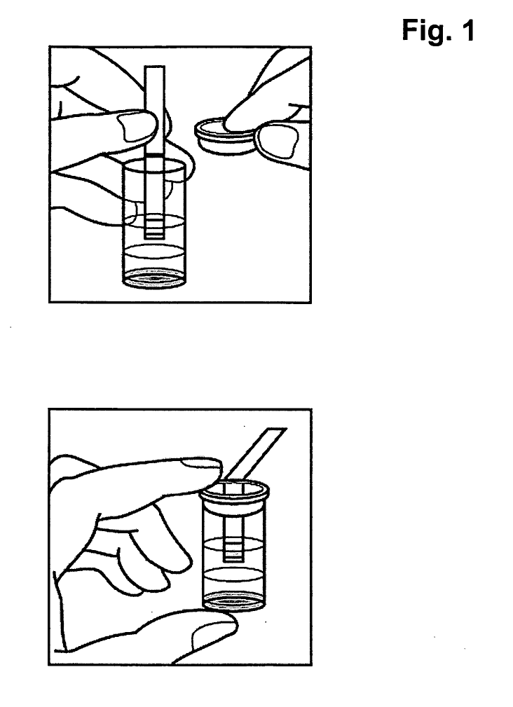

FIG. 1 shows a diagrammatic representation of the analytical vessel and the positioning of the test stick.

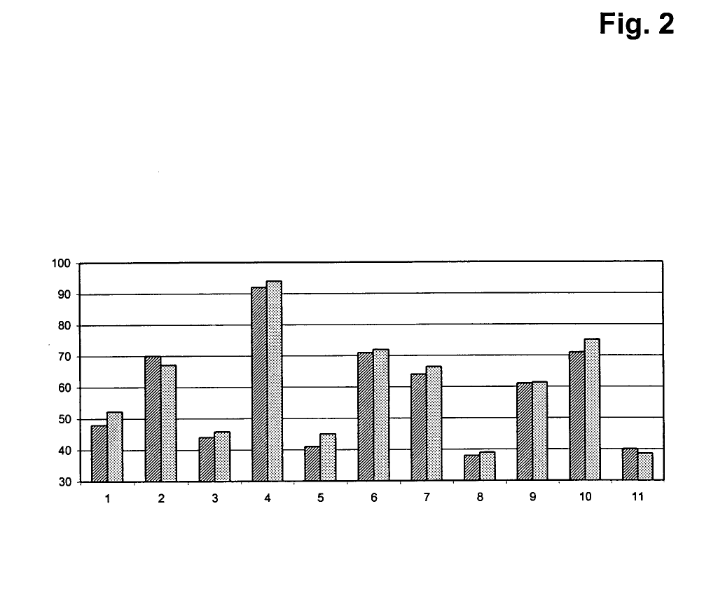

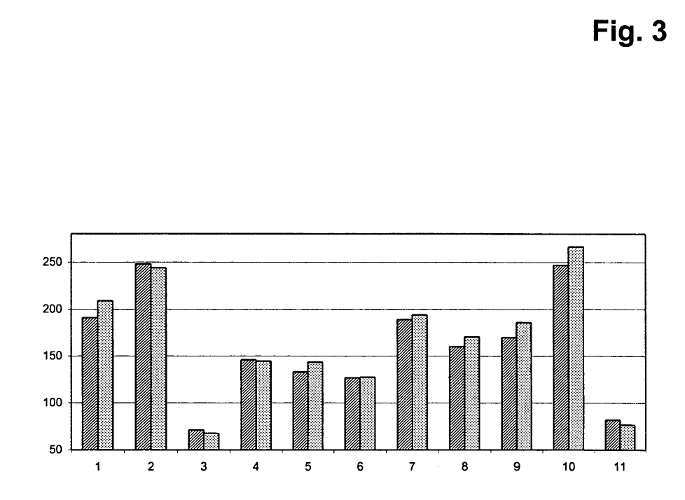

FIGS. 2 and 3 show the result of a comparison of the method according to the invention with a photometric determination. Further details are given in Examples 2 and 3.

In accordance with the invention, all liquid products obtainable from grapes are referred to as must and wine. The method according to the invention is specifically designed, with respect to its sensitivity and handling, for the particular requirements of must and wine since these, as described above, make particularly high demands of an analysis owing to their composition (colour, pH, etc.). However, the method according to the invention can of course also be employed for the determination of corresponding nitrogen components in other aqueous liquids. These liquids are therefore also encompassed in accordance with the invention by the term must and wine.

The determination system, i.e. the test stick, is in the form of an impregnated matrix, i.e. all reagents necessary for the selective determination of ammonium (colouring reagent, buffer system, optionally also stabilisers and solubilisers) are embedded in a sorptive support. The resultant colour reaction is evaluated reflectometrically or visually by comparison with a colour card.

The test stick according to the invention determines nitrogen in the form of ammonium. On the one hand free ammonium and on the other hand ammonium which has previously been liberated from arginine can be determined here. Differentiation of the source of the yeast-available nitrogen is thus additionally possible.

The liberation of ammonium from arginine is carried out by an enzymatic route corresponding to a method in accordance with the following known reaction scheme:
Degradation of the arginine by the enzyme arginase to give urea 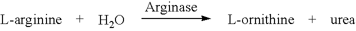
Degradation of the urea by the enzyme urease to give ammonia

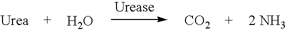

The prerequisite for simple performance of the determination is the possibility of simple addition of the enzymes to the sample. The most suitable metering form is the addition in the form of drops.

It has been found that an enzymatic reaction is not possible directly in the original wine matrix since interference by wine constituents or by the acidic wine matrix occurs. Large pre-dilutions of the wine are not possible since the requisite sensitivity of the determination system is thereby no longer ensured. It has been found that dilution of the wine in the ratio 1:1 to 1:5 with an alkaline buffer system of pH 9-10 facilitates a good reaction. A triethanolamine hydrochloride/NaOH buffer, in particular a triethanolamine hydrochloride/NaOH buffer of pH 9.35 has proven particularly suitable (preparation see practical test 2). Higher dilutions than 1:5 are not appropriate owing to the loss in sensitivity, preference is given to dilutions in the ratio of about 1:1.

For the determination of total ammonium (sum of free ammonium and ammonium from arginine), the sample is therefore pretreated by pre-dilution of the wine with an alkaline buffer and addition of the two enzymes before insertion of the test stick.

For this purpose, the enzymes are typically each in aqueous solution. Preference is given to solutions in which the respective enzyme remains stable and active for a particularly long time.

The urease solution is preferably a buffered glycerine solution (pH about 6.1). The urease activity should be in the range 10-30 U/l, preferably about 20 U/l, per determination.

The arginase solution is preferably a manganese-containing maleic acid buffer (pH about 7.0). The arginase activity should be in the range 3-10 U/l, preferably about 5 U/l, per determination.

The entire reaction should be carried out in the range 19-23° C., otherwise deviations of greater than 10% may occur. The duration of the incubation of the sample with the enzyme solutions is typically 15 minutes to several hours, preferably 20 to 30 minutes.

It has been found that the analysis of the ammonium cannot be carried out directly in the sample solution, i.e. in the must and wine, but instead is only possible in the gas phase above the sample.

To this end, use is made of a test stick which has the determination reagents for ammonium or ammonia.

The determination system can be all ammonium-selective determination reactions. Particular preference is given to the known determination using “Nessler's reagent” (K₂Hgl₄), as described below (Jander, Blasius: Lehrbuch der analytischen and präparativen anorganischen Chemie [Textbook of Analytical and Preparative Organic Chemistry] (1979) Verlag Hirzel, Stuttgart).
NH₃+2 Hgl₄²⁻+3 OH⁻→Hg₂NI.H₂O+2 H₂O+7 I⁻

In this case, the test stick is treated with an impregnation solution comprising potassium iodide and mercury iodide. Suitable impregnation solutions have proven to be those which comprise 0.5 to 5% by weight, preferably about 1%, of the respective iodide. The solvent used is typically water or mixtures of water with organic solvents which are miscible therein, such as methanol.

Sorptive supports which can be used are all materials which are usually in use for such tests. The most widespread is the use of filter paper, but it is also possible to employ other sorptive cellulose or plastic products. The sorptive supports are impregnated in a known manner with impregnation solutions which comprise all reagents necessary for the determination. The impregnated and dried papers can be suitably cut to size and adhesively bonded or sealed to support films in a known manner.

In order to facilitate a reaction of the analyte in the gas phase with the reagent system of the test stick, the latter must be moistened before the reaction. Water has proven to be not very suitable for the moistening. Surprisingly, it has been found that moistening with a suitable reagent, depending on the determination reaction used, results in particularly high sensitivity and uniform coloration of the reaction field. In the case of determination using Nessler's reagent, dilute bases, in particular inorganic bases, such as NaOH or KOH, have proven suitable. Particular preference is given to dilute sodium hydroxide solution (0.05 to 0.5 mol/l, preferably about 0.05 to 0.1 mol/l).

Determination of ammonium via the liquid sample solution can be carried out directly using the moistened, ammonium-selective test stick. In order to achieve the requisite sensitivity of the determination system, the pH of the sample solution must be rendered alkaline. Preference is given to a pH>11 since at this pH ammonium ions are completely in the form of highly volatile ammonia. In principle, all strong bases or buffer systems are suitable for the alkalinisation. 32% sodium hydroxide solution has proven particularly suitable.

FIG. 1 shows a possible embodiment of the determination system according to the invention. For the analysis, the test stick here is introduced into the gas space above the sample and fixed by means of a lid. In a preferred embodiment, the suitable separation of the test strip from the sample solution is indicated, as shown in FIG. 1, by, for example, ring-shaped markings on the analytical vessel.

By changing the size and shape of the analytical vessel used, the position of the test stick during the determination and the amount of sample used, it is possible to influence the sensitivity of the determination system and the reproducibility of the determination, albeit only slightly.

Particularly advantageous vessels have proven to be those having a volume of 15-50 ml and amounts of sample of 0.5-1.0 ml.

In order to avoid undesired contact of the reaction zone of the test stick with the sample solution, the test stick must be positioned at a sufficiently large separation above the sample. In a preferred embodiment, a marking on the test stick indicates how far it should be introduced into the analytical vessel.

The method according to the invention accordingly comprises the following method steps:
a) Provision of a test kit according to the invention, at least consisting of test strip and sealable analytical vessel
b) Addition of the sample solution to the analytical vessel. The sample solution here can be the pure must or wine or, in particular for the determination of total ammonium, must or wine which has been diluted with an alkaline buffer system of pH 9-10 as described above.
c) Optionally addition of the enzymes arginase and urease in order to liberate ammonium from arginine, and incubation. This step is only carried out for the determination of total ammonium.
d) Alkalinisation of the sample solution in order to expel the ammonium as ammonia
e) Introduction of the moistened test stick into the gas space above the sample and sealing of the analytical vessel
f) Qualitative and/or quantitative analysis of the colour development on the test strip. The analysis is carried out after a brief incubation time of a few minutes, typically 3 to 8 minutes, preferably about 5 minutes.

The test kit according to the invention contains at least one test stick for the determination of ammonium in the gas phase and a sealable analytical vessel. Further optional constituents are solutions for moistening the test stick, for dilution of the sample solution or for alkalinisation of the sample solution. The test kit may equally contain the enzymes arginase and/or urease in solid or dissolved form.

In a preferred embodiment, the test stick of the test kit is impregnated with Nessler's reagent, i.e. with potassium iodide and mercury iodide.

In a further preferred embodiment, the test kit additionally contains one or more of the following components: 0.1 mol/l NaOH solution for moistening the test stick, 32% NaOH solution for expelling the ammonium, alkaline buffer solution for dilution of the sample solution, and the two enzymes arginase and urease in dissolved form.

Besides the great sensitivity of the test kit and its simple handling, a further advantage is its long shelf life. Both the test stick and also the buffer solutions are stable for several months to years. Even the enzyme solutions are stable for several months if they have been prepared, for example, in accordance with the details in Example 3.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application EP 04 009 399.9, filed on 21.04.2004, is incorporated into this application by way of reference.

### EXAMPLES

### Example 1

Determination of Free Ammonium in Grape Must/Wine—Reflectometric Evaluation of the Reaction Colour:

Production of the Test Sticks:

The following impregnation solution is applied to a filter paper (for example Binzer, 1450 CV; acid-washed) and then dried using warm air. The paper is sealed onto a white support film using hot-melt adhesive (for example Dynapol S 1272 adhesive) and cut suitably into strips, so that a reaction zone of about 6 mm×8 mm results.

Composition of the Impregnation Solution:

Potassium iodide and mercury iodide are dissolved in a water/methanol mixture (1:1) so that a 1% solution is present in each case.

Analysis:

| | | | |
|---|---|---|---|
| 1) Prepared sample solution | 1.0 | ml | Introduce into the test vessel |
| | | | (about 20 ml) |
| 3) NaOH, 0.1 mol/l | 1 | drop | Moisten the reaction zone of |
| | | | the analytical stick and tip |
| | | | excess drops off on an all- |
| | | | purpose cloth |
| | | | Open test vessel and add |
| | | | dropwise. |
| 3) 32% NaOH | 5 | drops | Immediately seal lid of the |
| | | | measurement vessel with |
| | | | suitably fixed sticks (see figure) |

Depending on the degree of ammonium in the sample solution, a yellow-brown coloration forms which can be evaluated reflectometrically or by comparison with a colour card after 3-5 minutes. In

For quantitative evaluation, the test strips are, after a suitable reaction time, evaluated in a small hand diode-based reflectometer (RQflex® reflectometer). The correlation between the measured relative remission (%) and the content of ammonium is shown by Table 1.

TABLE 1

| NH₄ (mg/l) | % rem |
|---|---|
| 0 | 75 |
| 15 | 70 |
| 25 | 65 |
| 50 | 55 |
| 75 | 45 |
| 100 | 40 |
| 150 | 35 |

### Example 2

Practical Test 1:

Various wine samples were investigated using the method according to the invention and the result compared with the photometric method.

The result is shown in FIG. 2, where the cross-hatched bars show the result of the reflectometric analysis according to the invention and the dotted bars show that of the photometric analysis.

Samples 1 to 11 are plotted on the abscissa, and the NH₃ concentration in mg/l is plotted on the ordinate. It can be seen that the significantly simpler method according to the invention is just as sensitive and accurate as the photometric method.

### Example 3

Practical Test 2:

Determination of Total Ammonium in Grape Must/Wine—Reflectometric Evaluation of the Reaction Colour:

Buffer solution for adjustment of the pH

100 g of triethanolamine hydrochloride are dissolved In 250 ml of deionised water, and about 200 g of sodium hydroxide solution (10%) are added. The pH is adjusted to 9.35 using NaOH.

The Following Solutions are Employed for the Liberation of Ammonium from Arginine:

Urease Solution:

A solution of water and glycerine (1:1) is adjusted to about pH 6.1 using NaOH (1 mol/l). Urease is dissolved therein. The urease activity is about 20 U per determination.

Arginase Solution

The arginase solution is prepared as described by Bergmeyer (J. Bergmeyer: Methods of Enzymatic Analysis (1983), Volume 2, Verlag Chemie, Weinheim).

The arginase activity is about 5 Upper determination.

The test sticks are produced as described in Example 1.

Analysis:

| | | | |
|---|---|---|---|
| 1) Prepared sample solution | 1.0 | ml | Introduce into the test |
| | | | vessel (about 20 ml) |
| 2) Buffer solution | 1.0 | ml | add and mix |
| 3) Arginase solution | 3 | drops | add dropwise |
| 4) Urease solution | 1 | drop | add dropwise and leave to |
| | | | stand for 20 minutes |
| 5) NaOH, 0.1 mol/l | 1 | drop | moisten reaction zone of |
| | | | the analytical stick and |
| | | | tip excess drops off on an |
| | | | all-purpose cloth. |
| 6) 32% NaOH | 5 | drops | open test vessel and add |
| | | | dropwise. |
| | | | Immediately seal lid of |
| | | | the measurement vessel |
| | | | with suitably fixed |
| | | | sticks (see figure) |

Depending on the degree of ammonium in the sample solution, a yellow-brown coloration forms which can be evaluated reflectometrically or by comparison with a colour card after 3-5 minutes.

The result is shown in FIG. 3, where the cross-hatched bars show the result of the reflectometric analysis according to the invention and the dotted bars show that of the photometric analysis.

Samples 1 to 11 are plotted on the abscissa and the NH₃ concentration in mg/l is plotted on the ordinate. It can be seen that the significantly simpler method according to the invention is just as sensitive and accurate as the photometric method.

### Example 4

Practical test 3:

Determination of Free Ammonium and Total Ammonium in Grape Must/Wine—Checking of the Recovery in Doping Experiments Compared with Photometric Test

Doping table: All data in mg/l

| | Addition of free ammonium or arginine | | |
|---|---|---|---|
| Sample No. | FA | Arginine | TA (calculated) |
| 1 | 40 | 10 | 59 |
| 2 | 40 | 20 | 84 |
| 3 | 40 | 40 | 123 |
| 4 | 60 | 10 | 84 |
| 5 | 60 | 20 | 103 |
| 6 | 60 | 40 | 144 |
| 7 | 80 | 10 | 102 |
| 8 | 80 | 20 | 122 |
| 9 | 85 | 40 | 163 |

FA = free ammonium; TA = total ammonium

Results table: All data in mg/l

| | Results of photometric test | | | Results of test-strip reflectometry | | |
|---|---|---|---|---|---|---|
| Sample | | Ferm N | TA | | Ferm N | |
| No. | FA | value | (calculated) | FA | (calculated) | TA |
| 1 | 43 | 9 | 61 | 44 | 12 | 68 |
| 2 | 45 | 20 | 81 | 44 | 16 | 78 |
| 3 | 44 | 40 | 123 | 45 | 26 | 99 |
| 4 | 65 | 9 | 81 | 59 | 17 | 93 |
| 5 | 65 | 19 | 101 | 63 | 21 | 106 |
| 6 | 66 | 40 | 143 | 66 | 35 | 137 |
| 7 | 83 | 10 | 101 | 76 | 15 | 107 |
| 8 | 84 | 19 | 121 | 79 | 20 | 119 |
| 9 | 85 | 40 | 163 | 80 | 32 | 145 |

Ferm N value = (T-NH₄ − F-NH₄) × 0.483

1. Test kit for the determination of yeast-available nitrogen in must and wine, at least consisting of a test strip for the determination of ammonia in the gas phase and a sealable vessel.

2. Test kit according to claim 1, characterised in that the test strip of the kit comprises potassium iodide and mercury iodide as determination reagent.

3. Test kit according to claim 1, characterised in that the test kit contains a test strip which has been impregnated with 0.5 to 5% solutions of potassium iodide and mercury iodide.

4. Test kit according to claim 1, characterised in that the test kit additionally contains one or more of the following components:
0.1 mol/l NaOH solution
32% NaOH solution
alkaline buffer solution for dilution of the sample solution
the two enzymes arginase and urease in dissolved form.

5. Method for the determination of yeast-available nitrogen in must and wine, characterised by the following method steps:
a) provision of a test kit according to claim 1,
b) addition of the sample solution to the analytical vessel,
c) optionally addition of the enzymes arginase and urease for liberation of ammonium from arginine, and incubation,
d) alkalinisation of the sample solution in order to expel the ammonium as ammonia,
e) introduction of the moistened test stick into the gas space above the sample and sealing of the analytical vessel,
f) qualitative and/or quantitative analysis of the colour development on the test strip.

6. Method according to claim 5, characterised in that a test stick moistened with 0.1 mol/l NaOH solution is employed in step e).

7. Method according to claim 1, characterised in that the sample is rendered alkaline using 32% NaOH solution in step d).

8. Method according to claim 1, characterised in that the quantitative analysis in step f) is carried out using a reflectometer.
